(19) United States
(12) Patent Application Publication
(10) Pub. No.: US 2024/0115208 A1
(43) Pub. Date: Apr. 11, 2024

(54) VITAL SIGN DETECTION DEVICE, VITAL SIGN DETECTION METHOD, AND AUTOMOTIVE DEVICE

(71) Applicant: Mitsubishi Electric Corporation, Tokyo (JP)

(72) Inventor: Shusaku TAKAMOTO, Tokyo (JP)

(73) Assignee: Mitsubishi Electric Corporation, Tokyo (JP)

(21) Appl. No.: 18/276,984

(22) PCT Filed: Mar. 26, 2021
(86) PCT No.: PCT/JP2021/012896

§ 371 Date: Aug. 11, 2023

(51) Int. Cl. A61B 5/00

(52) U.S. Cl. CPC A61B 5/6893; A61B 5/7203

(57) ABSTRACT

A vital sign detection device includes: a sensor signal acquiring unit to acquire a sensor signal representing a sensing result of a biosensor to sense a vital sign of a measurement subject; a spectrum calculating unit to calculate a frequency spectrum of the sensor signal; a frequency component identifying unit to identify one or more frequency components which may represent vital signs of the measurement subject, in a plurality of frequency components included in the frequency spectrum calculated; a noise band identifying unit to identify a noise band which is a noise-containing frequency band, from a noise signal representing an observation result of a noise observer to observe noise which may be mixed in the sensor signal representing the sensing result of the biosensor; and a noise removing unit to remove a frequency component which is included in the noise band identified, in the one or more frequency components identified.

### TECHNICAL FIELD

The present disclosure relates to a vital sign detection device and a vital sign detection method, and an automotive device including the vital sign detection device.

### BACKGROUND ART

There is a biosensor to sense vital signs of a measurement subject. The biosensor outputs sensor signals representing sensing results of the vital signs. The vital signs sensed by the biosensors include the respiratory rate of the measurement subject, the pulse rate of the measurement subject and the like. Noises mixed in the sensor signals output from the biosensor undesirably cause deterioration of the precision of the sensing results of vital signs in some cases.

As for a technology related to removal of noises mixed in sensor signals output from a biosensor, there is a bioinformation detection device disclosed in Patent Literature 1. The bioinformation detection device calculates a frequency spectrum of a sensor signal, and identifies one or more frequency components which may represent vital signs of a measurement subject in a plurality of frequency components included in the frequency spectrum. Then, the bioinformation detection device removes a frequency component in the one or more identified frequency components, the frequency component being greater than an assumed maximum value of frequency components representing vital signs or being smaller than an assumed minimum value of the frequency components representing the vital signs. Accordingly, the bioinformation detection device removes a noise frequency component if the frequency component is greater than the assumed maximum value or is smaller than the assumed minimum value.

### CITATION LIST

### Patent Literature

Patent Literature 1: JP 2019-126407 A

### SUMMARY OF INVENTION

### Technical Problem

The bioinformation detection device disclosed in Patent Literature 1 does not remove, from among the one or more frequency components which may represent the vital signs of the measurement subject, a frequency component which has a magnitude being equal to or smaller than the assumed maximum value and being equal to or greater than the assumed minimum value. Accordingly, there is a problem with the vital sign detection device that a frequency component with a magnitude which is equal to or smaller than the assumed maximum value and is equal to or greater than the assumed minimum value is not removed even when the frequency component is a noise frequency component.

The present disclosure has been made in order to solve the problem as described above, and an object thereof is to obtain a vital sign detection device and a vital sign detection method that make it possible to remove a noise frequency component with a magnitude which is equal to or smaller than an assumed maximum value and is equal to or greater than an assumed minimum value.

### Solution to Problem

A vital sign detection device according to the present disclosure includes: a sensor signal acquiring unit to acquire a sensor signal representing a sensing result of a biosensor to sense a vital sign of a measurement subject; a spectrum calculating unit to calculate a frequency spectrum of the sensor signal acquired by the sensor signal acquiring unit; a frequency component identifying unit to identify one or more frequency components which may represent vital signs of the measurement subject, from among a plurality of frequency components included in the frequency spectrum calculated by the spectrum calculating unit; a noise band identifying unit to identify a noise band which is a frequency band containing noise, from a noise signal representing an observation result of a noise observer to observe the noise which may be mixed in the sensor signal representing the sensing result of the biosensor; and a noise removing unit to remove a frequency component which is included in the noise band identified by the noise band identifying unit, from among the one or more frequency components identified by the frequency component identifying unit.

### Advantageous Effects of Invention

The present disclosure makes it possible to remove a noise frequency component with a magnitude which is equal to or smaller than an assumed maximum value and is equal to or greater than an assumed minimum value.

### BRIEF DESCRIPTION OF DRAWINGS

FIG. 1 is a configuration diagram depicting an automotive device including a vital sign detection device 3 according to a first embodiment.

FIG. 2 is a configuration diagram of the vital sign detection device 3 according to the first embodiment.

FIG. 3 is a hardware configuration diagram depicting hardware of the vital sign detection device 3 according to the first embodiment.

FIG. 4 is a hardware configuration diagram of a computer in a case where the vital sign detection device 3 is implemented by software, firmware or the like.

FIG. 5 is a flowchart depicting a vital sign detection method which is a process procedure performed in the vital sign detection device 3.

FIG. 6A is an explanatory diagram depicting a frequency spectrum including a respiratory-rate frequency component and a pulse-rate frequency component as frequency components of vital signs of a measurement subject, and FIG. 6B is an explanatory diagram depicting a frequency spectrum including a noise frequency component in addition to the respiratory-rate frequency component and the pulse-rate frequency component.

FIG. 7 is an explanatory diagram depicting the frequency components that are remaining even after the frequency component included in a noise band is removed by a noise removing unit 15.

FIG. 8 is a configuration diagram depicting an automotive device including a vital sign detection device 3 according to a second embodiment.

FIG. 9 is a configuration diagram of the vital sign detection device 3 according to the second embodiment.

FIG. 10 is a hardware configuration diagram depicting hardware of the vital sign detection device 3 according to the second embodiment.

### DESCRIPTION OF EMBODIMENTS

Hereinbelow, modes for carrying out the present disclosure are explained with reference to attached figures in order to explain the present disclosure in more detail.

### First Embodiment

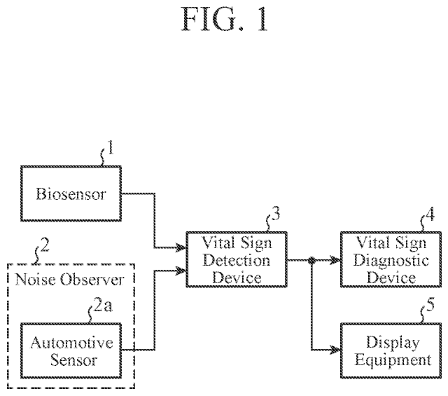

FIG. 1 is a configuration diagram depicting an automotive device including a vital sign detection device 3 according to a first embodiment.

The automotive device depicted in FIG. 1 includes a biosensor 1, a noise observer 2, the vital sign detection device 3, a vital sign diagnostic device 4 and display equipment 5.

The vital sign detection device 3 depicted in FIG. 1 is mounted on the automotive device. The vital sign detection device 3 is not limited to one mounted on an automotive device, but may be one that is installed at a hospital, a public facility or the like.

The automotive device is mounted on a vehicle which a measurement subject has gotten in.

For example, the biosensor 1 is implemented by video equipment or a radio wave sensor.

The biosensor 1 senses vital signs of the measurement subject, and outputs a sensor signal representing a sensing result to the vital sign detection device 3.

The noise observer 2 observes noise which may be mixed in the sensor signal representing the sensing result of the biosensor 1.

The noise observer 2 outputs a noise signal representing a noise observation result to the vital sign detection device 3.

In the vital sign detection device 3 depicted in FIG. 1, the noise observer 2 includes an automotive sensor 2a.

The automotive sensor 2a is implemented by a yaw rate sensor, an acceleration sensor or the like of the vehicle.

The automotive sensor 2a observes a vibration of the vehicle which the measurement subject has gotten in, as noise which may be mixed in the sensor signal.

The automotive sensor 2a outputs, as the noise signal, a vibration signal representing the vibration of the vehicle to the vital sign detection device 3.

Note that the yaw rate sensor is a sensor to detect the advancing direction of the vehicle. The vehicle vibrates along with changes of the advancing direction. Accordingly, vibrations of the vehicle can be observed by analyzing sensing results of the yaw, rate sensor.

The acceleration sensor is a sensor to detect the acceleration of the vehicle. The vehicle vibrates along with changes of the acceleration. Accordingly, vibrations of the vehicle can be observed by analyzing sensing results of the acceleration sensor.

The vital sign detection device 3 detects vital signs of the measurement subject on the basis of the sensor signal output from the biosensor 1.

The vital sign diagnostic device 4 makes a diagnosis about the measurement subject on the basis of the vital signs detected by the vital sign detection device 3.

The display equipment 5 causes the vital signs of the measurement subject detected by the vital sign detection device 3 to be displayed by a display.

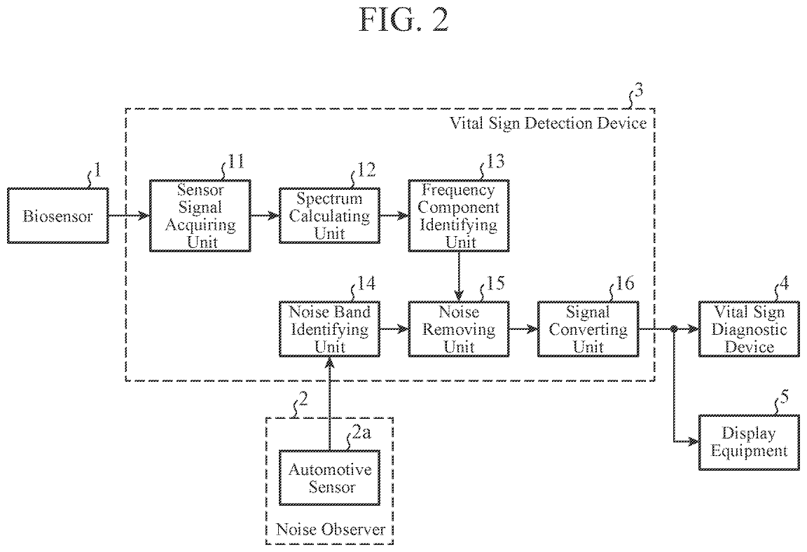

FIG. 2 is a configuration diagram depicting the vital sign detection device 3 according to the first embodiment.

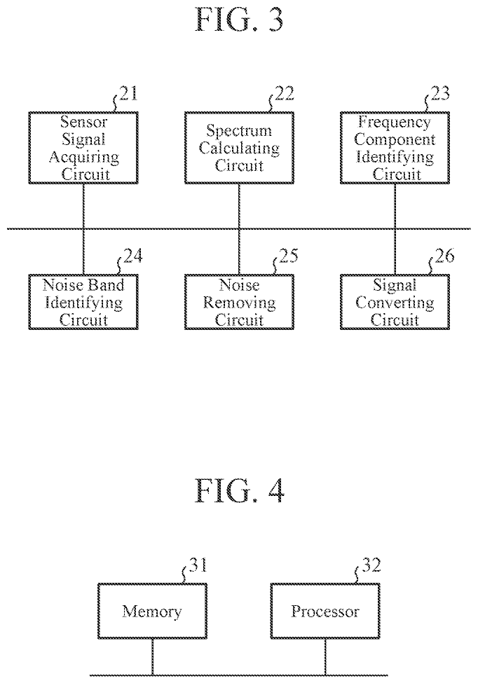

FIG. 3 is a hardware configuration diagram depicting hardware of the vital sign detection device 3 according to the first embodiment.

The vital sign detection device 3 depicted in FIG. 2 includes a sensor signal acquiring unit 11, a spectrum calculating unit 12, a frequency component identifying unit 13, a noise band identifying unit 14, a noise removing unit 15 and a signal converting unit 16.

For example, the sensor signal acquiring unit 11 is implemented by a sensor signal acquiring circuit 21 depicted in FIG. 3.

The sensor signal acquiring unit 11 acquires the sensor signal output from the biosensor 1.

The sensor signal acquiring unit 11 outputs the sensor signal to the spectrum calculating unit 12.

For example, the spectrum calculating unit 12 is implemented by a spectrum calculating circuit 22 depicted in FIG. 3.

The spectrum calculating unit 12 calculates a frequency spectrum of the sensor signal acquired by the sensor signal acquiring unit 11. For example, the spectrum calculating unit 12 calculates the frequency spectrum by performing FFT (Fast Fourier Transform) on the sensor signal.

The spectrum calculating unit 12 outputs the frequency spectrum to the frequency component identifying unit 13.

For example, the frequency component identifying unit 13 is implemented by a frequency component identifying circuit 23 depicted in FIG. 3.

The frequency component identifying unit 13 identifies one or more frequency components which may represent vital signs of the measurement subject, from among a plurality of frequency components included in the frequency spectrum calculated by the spectrum calculating unit 12.

That is, the frequency component identifying unit 13 compares each frequency component included in the frequency spectrum with a first threshold Th₁. The frequency component identifying unit 13 identifies, from among the plurality of frequency components, a frequency component which is greater than the first threshold Th₁, as a frequency component which may represent a vital sign of the measurement subject. The first threshold Th₁ may be stored in an internal memory of the frequency component identifying unit 13 or may be given from the outside of the vital sign detection device 3.

The frequency component identifying unit 13 outputs one or more identified frequency components to the noise removing unit 15.

For example, the noise band identifying unit 14 is implemented by a noise band identifying circuit 24 depicted in FIG. 3.

The noise band identifying unit 14 acquires the noise signal from the noise observer 2, and identifies a noise band which is a noise-containing frequency band on the basis of the noise signal.

That is, the noise band identifying unit 14 acquires the vibration signal as the noise signal from the automotive sensor 2a. The noise band identifying unit 14 calculates a frequency spectrum of the vibration signal, and identifies a band of frequencies representing a vibration of the vehicle as the noise band from the frequency spectrum of the vibration signal.

The noise band identifying unit 14 outputs the noise band to the noise removing unit 15.

For example, the noise removing unit 15 is implemented by a noise removing circuit 25 depicted in FIG. 3.

The noise removing unit 15 removes a frequency component which is included in the noise band identified by the noise band identifying unit 14, from among the one or more frequency components identified by the frequency component identifying unit 13.

The noise removing unit 15 outputs, to the signal converting unit 16, frequency components that are remaining even after the frequency component included in the noise band is removed, from among the one or more frequency components identified by the frequency component identifying unit 13.

For example, the signal converting unit 16 is implemented by a signal converting circuit 26 depicted in FIG. 3.

The signal converting unit 16 converts, into a time-domain signal, the frequency components that are remaining without being removed by the noise removing unit 15, from among the one or more frequency components identified by the frequency component identifying unit 13. For example, the signal converting unit 16 converts the remaining frequency components into the time-domain signal by performing inverse FFT on the remaining frequency components. The time-domain signal is equivalent to a signal obtained by removing the noise from the sensor signal acquired by sensor signal acquiring unit 11.

The signal converting unit 16 outputs the sensor signal after the noise removal, to the vital sign diagnostic device 4 or the display equipment 5.

It is assumed in FIG. 2 that each of the sensor signal acquiring unit 11, the spectrum calculating unit 12, the frequency component identifying unit 13, the noise band identifying unit 14, the noise removing unit 15 and the signal converting unit 16 which are constituent elements of the vital sign detection device 3, is implemented by dedicated hardware as depicted in FIG. 3. That is, it is assumed that the vital sign detection device 3 is implemented by the sensor signal acquiring circuit 21, the spectrum calculating circuit 22, the frequency component identifying circuit 23, the noise band identifying circuit 24, the noise removing circuit 25 and the signal converting circuit 26.

Each of the sensor signal acquiring circuit 21, the spectrum calculating circuit 22, the frequency component identifying circuit 23, the noise band identifying circuit 24, the noise removing circuit 25 and the signal converting circuit 26 is equivalent to, for example, a single circuit, a composite circuit, a programmed processor, a parallel-programmed processor, an Application Specific Integrated Circuit (ASIC), a Field-Programmable Gate Array (FPGA) or a combination of these.

The constituent elements of the vital sign detection device 3 are not limited to those implemented by dedicated hardware, but the vital sign detection device 3 may be implemented by software, firmware or a combination of software and firmware.

Software or firmware is stored as a program in a memory of a computer. The computer means hardware to execute the program, and is equivalent to, for example, a Central Processing Unit (CPU), a central processor, a processing unit, a computing device, a microprocessor, a microcomputer, a processor or a Digital Signal Processor (DSP).

FIG. 4 is a hardware configuration diagram of the computer in a case where the vital sign detection device 3 is implemented by software, firmware or the like.

In a case where the vital sign detection device 3 is implemented by software, firmware or the like, a program for causing the computer to execute a process procedure performed in each of the sensor signal acquiring unit 11, the spectrum calculating unit 12, the frequency component identifying unit 13, the noise band identifying unit 14, the noise removing unit 15 and the signal converting unit 16 is stored in a memory 31. Then, a processor 32 of the computer executes the program stored in the memory 31.

In addition, FIG. 3 depicts an example in which each of the constituent elements of the vital sign detection device 3 is implemented by dedicated hardware, and FIG. 4 depicts an example in which the vital sign detection device 3 is implemented by software, firmware or the like. However, these are merely examples. Some constituent elements of the vital sign detection device 3 may be implemented by dedicated hardware, and the remaining constituent elements may be implemented by software, firmware or the like.

Next, operation performed in the automotive device depicted in FIG. 1 is explained.

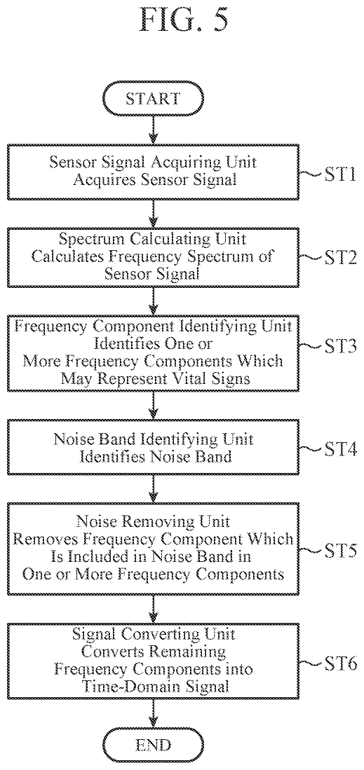

FIG. 5 is a flowchart depicting a vital sign detection method which is a process procedure performed in the vital sign detection device 3.

The biosensor 1 senses vital signs of a measurement subject, and outputs a sensor signal representing a sensing result to the vital sign detection device 3.

In a case where the biosensor 1 is implemented by video equipment, the biosensor 1 captures images of the measurement subject, and senses the vital signs of the measurement subject on the basis of a video of the measurement subject.

When the skin of the measurement subject is irradiated with light, the light radiated onto the skin is split into light specularly reflected on the skin of the measurement subject, light diffusedly reflected on the skin of the measurement subject and light scattered on the skin of the measurement subject.

The light scattered on the skin of the measurement subject includes scattered light that has been transmitted through the skin and reached blood vessels, and then exits from the surface of the skin again. Such scattered light includes information about pulse rate.

A part of the light having reached blood vessels is absorbed by hemoglobin included in blood. The amount of hemoglobin included in blood changes along with the pulsation of a blood flow, and the amount of light absorbed in the blood vessels also changes depending on the pulsation. Accordingly, the scattered light has a component that changes depending on the pulsation.

The biosensor 1 implemented by the video equipment can estimate the pulse rate, by extracting the component that is included in the scattered light and changes depending on the pulsation, from a luminance value of the skin surface represented by the video of the measurement subject. A process of estimating the pulse rate from the luminance value of the skin surface is itself a known technology, and accordingly, a detailed explanation thereof is omitted.

In a case where the biosensor 1 is implemented by a radio wave sensor, the biosensor 1 irradiates the body surface of the measurement subject with a microwave, and receives a reflected wave which is a microwave after reflection on the body surface.

Since the body of a human is vibrating due to heartbeats or breathing, the phase of the reflected wave changes along with a lapse of time. The biosensor 1 can estimate the pulse rate and the respiratory rate by detecting phase changes of the reflected wave. A process of estimating the pulse rate or the like from the phase changes of the reflected wave is itself a known technology, and accordingly, a detailed explanation thereof is omitted.

The automotive sensor 2a observes a vibration of a vehicle which the measurement subject has gotten in, as noise which may be mixed in the sensor signal representing the sensing result of the biosensor 1.

The automotive sensor 2a outputs, as a noise signal, a vibration signal representing the vibration of the vehicle to the vital sign detection device 3.

The sensor signal acquiring unit 11 of the vital sign detection device 3 acquires the sensor signal from the biosensor 1 (Step ST1 in FIG. 5). The sensor signal includes frequency components representing vital signs of the measurement subject. In addition, a noise frequency component is mixed in the sensor signal in some cases.

The sensor signal acquiring unit 11 outputs the sensor signal to the spectrum calculating unit 12.

The spectrum calculating unit 12 acquires the sensor signal from the sensor signal acquiring unit 11.

For example, the spectrum calculating unit 12 calculates a frequency spectrum of the sensor signal by performing FFT on the sensor signal (Step ST2 in FIG. 5).

The spectrum calculating unit 12 outputs the frequency spectrum to the frequency component identifying unit 13.

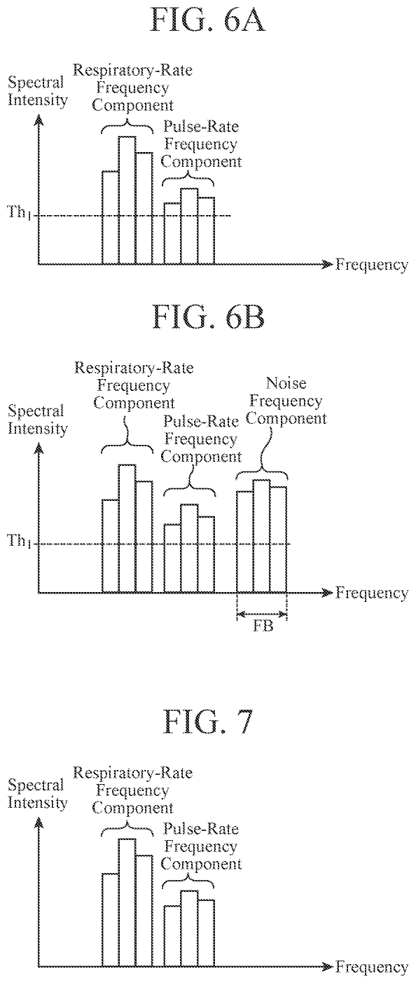

FIG. 6 includes explanatory diagrams depicting examples of the frequency spectrum calculated by the spectrum calculating unit 12.

FIG. 6A depicts a frequency spectrum including a respiratory-rate frequency component and a pulse-rate frequency component, as frequency components of vital signs of the measurement subject.

FIG. 6B depicts a frequency spectrum including a noise frequency component in addition to the respiratory-rate frequency component and the pulse-rate frequency component.

In FIG. 6A and FIG. 6B, the horizontal axes represent frequency, and the vertical axes represent spectral intensity.

In a case where the vehicle is vibrating, the vibration of the vehicle is mixed as noise in the sensor signal, and the noise frequency component as depicted in FIG. 6B is observed in the frequency spectrum in some cases.

The frequency component identifying unit 13 acquires the frequency spectrum from the spectrum calculating unit 12.

The frequency component identifying unit 13 identifies one or more frequency components which may represent vital signs of the measurement subject, from among a plurality of frequency components included in the frequency spectrum (Step ST3 in FIG. 5).

That is, the frequency component identifying unit 13 compares each frequency component included in the frequency spectrum with the first threshold Th₁. The frequency component identifying unit 13 identifies, from among the plurality of frequency components, a frequency component which is greater than the first threshold Th₁, as a frequency component which may represent a vital sign of the measurement subject.

In the example in FIG. 6B, the frequency component identifying unit 13 identifies the respiratory-rate frequency component, the pulse-rate frequency component and the noise frequency component, as frequency components which may represent vital signs of the measurement subject.

The frequency component identifying unit 13 outputs one or more identified frequency components to the noise removing unit 15.

The noise band identifying unit 14 acquires the vibration signal as the noise signal from the automotive sensor 2a.

For example, the noise band identifying unit 14 calculates a frequency spectrum of the vibration signal by performing FFT on the vibration signal.

The noise band identifying unit 14 identifies, as a noise band, a band of frequencies representing the vibration of the vehicle, from the frequency spectrum of the vibration signal (Step ST4 in FIG. 5).

The noise band identifying unit 14 outputs the noise band to the noise removing unit 15.

Hereinbelow, a noise band identification process performed in the noise band identifying unit 14 is explained specifically.

For example, the noise band identifying unit 14 calculates the frequency spectrum of the vibration signal by performing FFT on the vibration signal.

The noise band identifying unit 14 compares one or more frequency components included in the frequency spectrum of the vibration signal with a second threshold Th₂.

The noise band identifying unit 14 determines that among one or more frequency components included in the frequency spectrum of the vibration signal, a frequency band including a frequency component that is greater than the second threshold Th₂ is a band of frequencies representing the vibration of the vehicle, that is, the noise band. The second threshold Th₂ may be stored in an internal memory of the noise band identifying unit 14 or may be given from the outside of the vital sign detection device 3.

In the example in FIG. 6B, a frequency band FB is the noise band.

The noise removing unit 15 acquires, from the frequency component identifying unit 13, one or more frequency components which may represent vital signs of the measurement subject.

The noise removing unit 15 acquires the noise band from the noise band identifying unit 14.

The noise removing unit 15 removes, from among the one or more frequency components which may represent vital signs of the measurement subject, a frequency component which is included in the noise band (Step ST5 in FIG. 5).

The noise removing unit 15 outputs, to the signal converting unit 16, frequency components that are remaining even after the frequency component included in the noise band is removed, from among the one or more frequency components which may represent vital signs.

FIG. 7 is an explanatory diagram depicting the frequency components that are remaining even after the frequency component included in the noise band is removed by the noise removing unit 15.

In the example in FIG. 7, the noise which is the vibration of the vehicle is removed, and the respiratory-rate frequency component and the pulse-rate frequency component are remaining.

In FIG. 7, the horizontal axis represents frequency, and the vertical axis represents spectral intensity.

The signal converting unit 16 acquires the remaining frequency components from the noise removing unit 15.

The signal converting unit 16 converts the remaining frequency components into a time-domain signal by performing inverse FFT on the remaining frequency components (Step ST6 in FIG. 5). The time-domain signal is equivalent to a signal obtained by removing the noise from the sensor signal acquired by sensor signal acquiring unit 11.

The signal converting unit 16 outputs the sensor signal after the noise removal to the vital sign diagnostic device 4 or the display equipment 5.

When receiving the sensor signal after the noise removal from the vital sign detection device 3, the vital sign diagnostic device 4 makes a diagnosis about the measurement subject on the basis of the vital signs represented by the sensor signal after the noise removal.

When receiving the sensor signal after the noise removal from the vital sign detection device 3, the display equipment 5 causes the vital signs represented by the sensor signal after the noise removal to be displayed on the display.

In the first embodiment mentioned above, the vital sign detection device 3 is configured in such a manner that the vital sign detection device 3 includes: the sensor signal acquiring unit 11 to acquire a sensor signal representing a sensing result of the biosensor 1 to sense a vital sign of a measurement subject; the spectrum calculating unit 12 to calculate a frequency spectrum of the sensor signal acquired by the sensor signal acquiring unit 11; the frequency component identifying unit 13 to identify one or more frequency components which may represent vital signs of the measurement subject from among a plurality of frequency components included in the frequency spectrum calculated by the spectrum calculating unit 12; the noise band identifying unit 14 to identify a noise band which is a noise-containing frequency band, from a noise signal representing an observation result of the noise observer 2 to observe noise which may be mixed in the sensor signal representing the sensing result of the biosensor 1; and the noise removing unit 15 to remove a frequency component which is included in the noise band identified by the noise band identifying unit 14, from among the one or more frequency components identified by the frequency component identifying unit 13. Accordingly, the vital sign detection device 3 can remove a noise frequency component with a magnitude which is equal to or smaller than an assumed maximum value and is equal to or greater than an assumed minimum value.

### Second Embodiment

In a second embodiment, a vital sign detection device 3 in which a noise band identifying unit 17 acquires, as a noise signal, a physical motion signal representing a physical motion of a measurement subject observed by a measurement subject observer 2b, and identifies a noise band on the basis of the physical motion signal is explained.

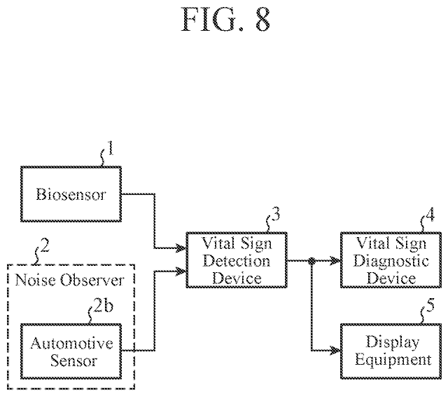

FIG. 8 is a configuration diagram depicting an automotive device including the vital sign detection device 3 according to the second embodiment. In FIG. 8, the same reference signs as in FIG. 1 represent the same or equivalent portions, and accordingly, detailed explanations thereof are omitted.

The automotive device depicted in FIG. 8 includes a biosensor 1, a noise observer 2, the vital sign detection device 3, a vital sign diagnostic device 4 and display equipment 5.

The vital sign detection device 3 depicted in FIG. 8 is mounted on the automotive device. The vital sign detection device 3 is not limited to one mounted on an automotive device, but may be one that is installed at a hospital, a public facility or the like.

In the vital sign detection device 3 depicted in FIG. 8, the noise observer 2 includes the measurement subject observer 2b.

For example, the measurement subject observer 2b is implemented by video equipment.

The measurement subject observer 2b captures images of the measurement subject, and, on the basis of video data of the measurement subject, observes a physical motion of the measurement subject as noise which may be mixed in a sensor signal.

The measurement subject observer 2b outputs the physical motion signal representing the physical motion of the measurement subject to the vital sign detection device 3.

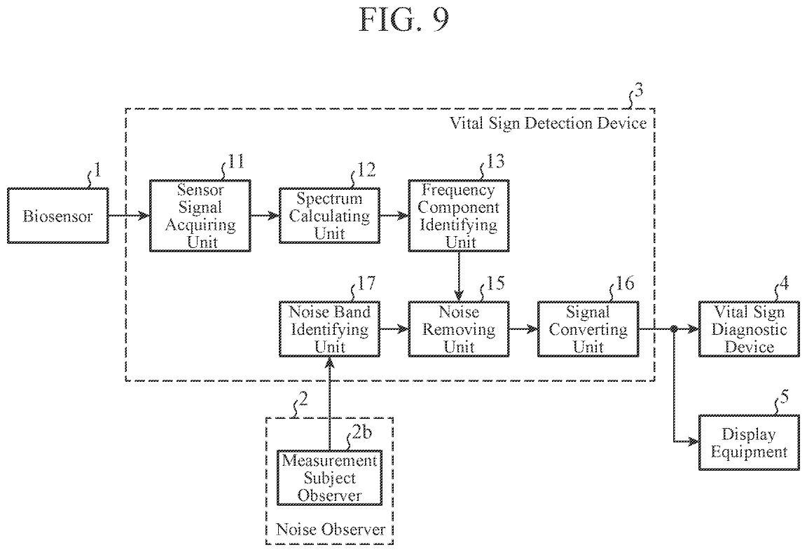

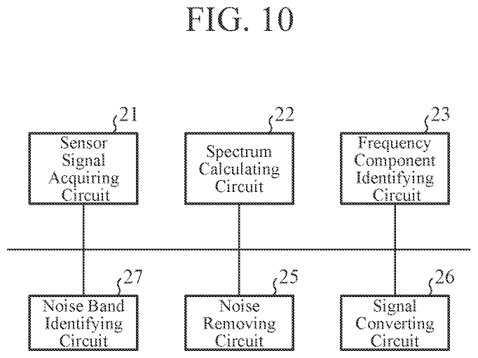

FIG. 9 is a configuration diagram of the vital sign detection device 3 according to the second embodiment.

FIG. 10 is a hardware configuration diagram depicting hardware of the vital sign detection device 3 according to the second embodiment.

In FIG. 9 and FIG. 10, the same reference signs as in FIG. 2 and FIG. 3 represent the same or equivalent portions, and accordingly, detailed explanations thereof are omitted.

The vital sign detection device 3 includes a sensor signal acquiring unit 11, a spectrum calculating unit 12, a frequency component identifying unit 13, the noise band identifying unit 17, a noise removing unit 15 and a signal converting unit 16.

For example, the noise band identifying unit 17 is implemented by a noise band identifying circuit 27 depicted in FIG. 10.

The noise band identifying unit 17 acquires the noise signal from the noise observer 2, and identifies a noise band which is a noise-containing frequency band from the noise signal.

That is, the noise band identifying unit 17 acquires the physical motion signal as the noise signal from the measurement subject observer 2b. The noise band identifying unit 17 identifies, as a noise band, a band of frequencies representing the physical motion on the basis of the physical motion signal.

The noise band identifying unit 17 outputs the noise band to the noise removing unit 15.

It is assumed in FIG. 9 that each of the sensor signal acquiring unit 11, the spectrum calculating unit 12, the frequency component identifying unit 13, the noise band identifying unit 17, the noise removing unit 15 and the signal converting unit 16 which are constituent elements of the vital sign detection device 3, is implemented by dedicated hardware as depicted in FIG. 10. That is, it is assumed that the vital sign detection device 3 is implemented by a sensor signal acquiring circuit 21, a spectrum calculating circuit 22, a frequency component identifying circuit 23, the noise band identifying circuit 27, a noise removing circuit 25 and a signal converting circuit 26.

Each of the sensor signal acquiring circuit 21, the spectrum calculating circuit 22, the frequency component identifying circuit 23, the noise band identifying circuit 27, the noise removing circuit 25 and the signal converting circuit 26 is equivalent to, for example, a single circuit, a composite circuit, a programmed processor, a parallel-programmed processor, an ASIC, an FPGA or a combination of these.

The constituent elements of the vital sign detection device 3 are not limited to those implemented by dedicated hardware, but the vital sign detection device 3 may be implemented by software, firmware or a combination of software and firmware.

In a case where the vital sign detection device 3 is implemented by software, firmware or the like, a program for causing the computer to execute a process procedure performed in each of the sensor signal acquiring unit 11, the spectrum calculating unit 12, the frequency component identifying unit 13, the noise band identifying unit 17, the noise removing unit 15 and the signal converting unit 16 is stored in the memory 31 depicted in FIG. 4. Then, the processor 32 depicted in FIG. 4 executes the program stored in the memory 31.

In addition, FIG. 10 depicts an example in which each of the constituent elements of the vital sign detection device 3 is implemented by dedicated hardware, and FIG. 4 depicts an example in which the vital sign detection device 3 is implemented by software, firmware or the like. However, these are merely examples. Some constituent elements of the vital sign detection device 3 may be implemented by dedicated hardware, and the remaining constituent elements may be implemented by software, firmware or the like.

Next, operation performed in the automotive device depicted in FIG. 8 is explained. It should be noted that since portions other than the measurement subject observer 2b and the noise band identifying unit 17 are similar to those in the automotive device depicted in FIG. 1, only operation performed in the measurement subject observer 2b and the noise band identifying unit 17 is explained here.

The measurement subject observer 2b captures images of the measurement subject, and, on the basis of video data of the measurement subject, observes a physical motion of the measurement subject as noise which may be mixed in a sensor signal representing a sensing result of the biosensor 1.

The measurement subject observer 2b outputs a physical motion signal representing the physical motion of the measurement subject to the vital sign detection device 3.

Since the body of a human is vibrating due to heartbeats or breathing, the physical motion of the measurement subject can be observed if the measurement subject observer 2b monitors the video data of the measurement subject.

The noise band identifying unit 17 acquires the physical motion signal as the noise signal from the measurement subject observer 2b.

The noise band identifying unit 17 identifies, as a noise band, a band of frequencies representing the physical motion on the basis of the physical motion signal.

That is, the noise band identifying unit 17 measures a change amount per unit time of a certain body part of the measurement subject on the basis of the physical motion signal. The certain body part may be the head, the chest, an arm or the like of the measurement subject.

From the change amount per unit time, the noise band identifying unit 17 identifies the band of frequencies representing the physical motion of the measurement subject. A process of identifying the band of frequencies representing the physical motion from the change amount per unit time is itself a known technology, and accordingly, a detailed explanation thereof is omitted.

The noise band identifying unit 17 outputs the noise band to the noise removing unit 15.

In the second embodiment mentioned above, the vital sign detection device 3 depicted in FIG. 9 is configured in such a manner that the noise band identifying unit 17 acquires, as a noise signal, a physical motion signal representing a physical motion of a measurement subject observed by the measurement subject observer 2b, and identifies a noise band on the basis of the physical motion signal. Accordingly, similarly to the vital sign detection device 3 depicted in FIG. 2, the vital sign detection device 3 depicted in FIG. 9 can remove a noise frequency component with a magnitude which is equal to or smaller than an assumed maximum value and is equal to or greater than an assumed minimum value.

In the vital sign detection device 3 depicted in FIG. 2, the noise band identifying unit 14 observes a vibration of a vehicle which a measurement subject has gotten in, as noise which may be mixed in a sensor signal representing a sensing result of the biosensor 1, on the basis of a vibration signal output from the automotive sensor 2a. In the vital sign detection device 3 depicted in FIG. 9, the noise band identifying unit 17 observes a physical motion of a measurement subject, as noise which may be mixed in a sensor signal, on the basis of a physical motion signal output from the measurement subject observer 2b.

However, these are merely examples. For example, the noise observer 2 of the automotive device may include noise measuring equipment to measure a frequency band of noise output from a power source or the like implemented on a vehicle, and the noise band identifying unit 14 or the noise band identifying unit 17 may acquire, as a noise band, the frequency band of the noise measured by the noise measuring equipment.

Note that the present disclosure allows any combinations of embodiments, modifications of any constituent elements in embodiments and omission of any constituent elements in embodiments.

### INDUSTRIAL APPLICABILITY

The present disclosure is suitable for a vital sign detection device and a vital sign detection method.

The present disclosure is suitable for an automotive device including the vital sign detection device.

### REFERENCE SIGNS LIST

1: biosensor, 2: noise observer, 2a: automotive sensor, 2b: measurement subject observer, 3: vital sign detection device, 4: vital sign diagnostic device, 5: display equipment, 11: sensor signal acquiring unit, 12: spectrum calculating unit, 13: frequency component identifying unit, 14, 17: noise band identifying unit, 15: noise removing unit, 16: signal converting unit, 21: sensor signal acquiring circuit, 22: spectrum calculating circuit, 23: frequency component identifying circuit, 24, 27: noise band identifying circuit, 25: noise removing circuit, 26: signal converting circuit, 31: memory, 32: processor

1. A vital sign detection device comprising:
sensor signal acquiring circuitry to acquire a sensor signal representing a sensing result of a biosensor to sense a vital sign of a measurement subject;
spectrum calculating circuitry to calculate a frequency spectrum of the sensor signal acquired by the sensor signal acquiring circuitry;
frequency component identifying circuitry to identify one or more frequency components which may represent vital signs of the measurement subject, from among a plurality of frequency components included in the frequency spectrum calculated by the spectrum calculating circuitry;
noise band identifying circuitry to identify a noise band which is a frequency band containing noise, from a noise signal representing an observation result of a noise observer to observe the noise which may be mixed in the sensor signal representing the sensing result of the biosensor; and
noise removing circuitry to remove a frequency component which is included in the noise band identified by the noise band identifying circuitry, from among the one or more frequency components identified by the frequency component identifying circuitry, wherein
the noise band identifying circuitry identifies, among one or more frequency components included in a frequency spectrum of the noise signal, a frequency band including a frequency component that is greater than a predetermined threshold, as the noise band.

2. The vital sign detection device according to claim 1, wherein
the noise observer includes an automotive sensor to observe a vibration of a vehicle which the measurement subject has gotten in, as the noise which may be mixed in the sensor signal representing the sensing result of the biosensor, and
the noise band identifying circuitry acquires, as the noise signal, a vibration signal representing the vibration of the vehicle observed by the automotive sensor, and identifies the noise band on a basis of the vibration signal.

3. The vital sign detection device according to claim 1, wherein
the noise observer includes a measurement subject observer to observe a physical motion of the measurement subject, as the noise which may be mixed in the sensor signal representing the sensing result of the biosensor, and
the noise band identifying circuitry acquires, as the noise signal, a physical motion signal representing the physical motion of the measurement subject observed by the measurement subject observer, and identifies the noise band on a basis of the physical motion signal.

4. The vital sign detection device according to claim 1, comprising a signal converting circuitry to convert, into a time-domain signal, a frequency component which is remaining without being removed by the noise removing circuitry, from among the one or more frequency components identified by the frequency component identifying circuitry.

5. A vital sign detection method comprising:
acquiring a sensor signal representing a sensing result of a biosensor to sense a vital sign of a measurement subject;
calculating a frequency spectrum of the sensor signal acquired;
identifying one or more frequency components which may represent vital signs of the measurement subject, from among a plurality of frequency components included in the frequency spectrum calculated;
identifying a noise band which is a frequency band containing noise, from a noise signal representing an observation result of a noise observer to observe the noise which may be mixed in the sensor signal representing the sensing result of the biosensor; and
removing a frequency component which is included in the noise band identified, from among the one or more frequency components identified, wherein
among one or more frequency components included in a frequency spectrum of the noise signal, a frequency band including a frequency component that is greater than a predetermined threshold is identified, as the noise band.

6. An automotive device comprising:
a biosensor to sense a vital sign of a measurement subject;
a noise observer to observe noise which may be mixed in a sensor signal representing a sensing result of the biosensor; and
the vital sign detection device according to claim 1.
